(19) United States
(12) Patent Application Publication
(10) Pub. No.: US 2011/0162437 A1
(43) Pub. Date: Jul. 7, 2011

(54) Biomarker for Mitochondrial Toxicity Associated with Phospholipidosis

(76) Inventors: Lucette Doessegger, Basel (CH); Goetz Schlotterbeck, Efringen-Kirchen (DE)

(21) Appl. No.: 12/443,986

(22) PCT Filed: Oct. 2, 2007
(86) PCT No.: PCT/EP07/08548

§ 371 Date: Dec. 7, 2009

(30) Foreign Application Priority Data
Oct. 9, 2006 (EP) 06121945.7

(51) Int. Cl. G01R 33/465; G01N 33/48

(52) U.S. Cl. 73/61.43; 324/307

(57) ABSTRACT

The present invention relates to a method for determining a risk of a phospholipidotic compound for inducing mitochondrial toxicity which is associated with drug-induced phospholipidosis, comprising a) measuring the level of PAG in a body fluid, b) comparing the level of determined PAG with the level of PAG of a control wherein an increased level of PAG in comparison with that control is indicative that said compound induces mitochondrial toxicity which is associated with drug-induced phospholipidosis.

This application is the National Stage of International Application No. PCT/EP2007/008548 filed Oct. 2, 2007, which claims the benefit of EP 06121945.7, filed Oct. 9, 2006, which is hereby incorporated by reference in its entirety.

For metabolic disorders such as drug induced phospholipidosis or phenylketonuria means for specific diagnosis are known.

However, the potential risk of these disorders for associated toxicities such as mitochondrial toxicity in humans is difficult to predict and the significance is unknown.

Mitochondria play a critical role in generating most of the cell's energy as ATP. They are also involved in other metabolic processes such as urea generation, haem synthesis and fatty acid beta oxidation.

Disruption of mitochondrial functions by drugs can result in cell death by apoptosis (K Chan et all, Drug-induced mitochondrial toxicity. Expert Opin. Drug Metab. Toxicol (2005) 1: (4):655-669).

Thus, there is a strong interest in a method for predicting mitochondrial toxicity associated with a metabolic disorder.

Therefore, the present invention provides a method for determining a risk of a phospholipidotic compound for inducing mitochondrial toxicity which is associated with a metabolic disorder, comprising
a) measuring the level of PAG in a body fluid sample,
b) comparing the level of determined PAG with the level of PAG of a control wherein an increased level of PAG in comparison with said control is indicative that said compound induces mitochondrial toxicity which is associated with a metabolic disorder.

Preferably, the metabolic disorders is a drug-induced phospholipidosis or a metabolic disorder caused by inborn errors such as e.g. inborn error of ureagenesis, or an inherited metabolic disorders such as e.g. phenylketonuria.

A preferred embodiment of the invention is therefore a method for determining a risk of a phospholipidotic compound for inducing mitochondrial toxicity which is associated with drug-induced phospholipidosis, comprising
a) measuring the level of PAG in a body fluid sample,
b) comparing the level of determined PAG with the level of PAG of a control wherein an increased level of PAG in comparison with said control is indicative that said compound induces mitochondrial toxicity which is associated with drug-induced phospholipidosis.

Drug-induced phospholipidosis is a storage disorder characterized by accumulation of phospholipids within cells, i.e., in the lysosomes. Compounds inducing phospholipidosis are cationic, generally amphiphilic molecules which interfere with the phospholipid metabolism and turnover. Few drugs have been reported to cause phospholipidosis in humans.

The onset and the severity of phospholipidosis depend on cumulative exposure and administration regimen (continuous versus intermittent).

The presence of foamy macrophages at light microscopic level is indicative of phospholipidosis. However, the final diagnosis of phospholipidosis is based on ultrastructural changes (membranous lamellar inclusions bodies) in the lysosomes of various cell types, especially in lymphocytes, macrophages, and parenchymal cells.

Phospholipidosis is a term for several of the lysosomal storage diseases in which there is an abnormal accumulation of lipids in the reticuloendothelial cells. The term “drug-induced phospholipidosis” means a phospholipidosis attributed to the presence of a drug in the body. Such a drug is called a phospholipidotic compound. The term “phospholipidotic compound” refers to a compound that is able to induce phospholipidosis (see for example Reasor and Kacew, “Drug-induced Phospholipidosis: Are there functional consequences?” Exp Biol Med, 2001, 226: 825-30).

A control may be an animal not treated with a compound or an animal treated with another compound whereby this other compound is not toxic for mitochondria, or the treated animal before treatment with a phospholipidotic compound (pre-dose values within the same individual).

The term “PAG” as used herein refers herein to phenylacetylglycine in rodents and to any molecule equivalent to phenylacetylglycine in species other than rodents such as for example phenylacetylglutamine in human. The term “PAG” also includes salts of phenylacetylglycine and of molecule equivalents of phenylacetylglycine.

This method may be used for testing of the toxicity of therapeutic compounds. Therapeutic compounds are compounds which may be used for treatment or prevention of diseases and disorder. Preferably, such a test may be done with a rat or a mouse or human body fluid samples. The test may be done with body fluid samples of any animal if said animal has a phenylacetylglycine equivalent.

Preferably, the body fluid sample is blood or urine. More preferably, the body fluid sample is urine. The methods for obtaining samples of body fluids are known to the skilled in the art.

The person skilled in the art is familiar with different methods of measuring the level of PAG, in particular of phenylacetylglycine and phenylacetyglutamine. The term “level” relates to amount or concentration of PAG in an individual or a sample taken from an individual.

In the context of the present invention, amount also relates to concentration. It is evident, that from the total amount of a substance of interest in a sample of known size, the concentration of the substance can be calculated, and vice versa.

The term “measuring” according to the present invention relates to determining the amount or concentration, preferably semi-quantitatively or quantitatively. Measuring can be done directly.

Methods for detecting PAG are well known to skilled in the art. Preferred methods comprise NMR (i.e. single pulse NMR as described in Keun, H. C et al., (2002) Physiological variation and analytical reproducibility in metabonomic urinalysis (Chem. Res. Tox. 15, 1380-1386), Mass Spectrometry (MS), MS combined with chromatographic techniques, liquid chromatography-ultraviolet detection (LC-UV), Liquid chromatography with photodiode array detection (LC-DAD), Gas Chromatography (GC).

The present invention also provides a use of PAG as marker for mitochondrial toxicity. Preferred is the use of PAG as marker for mitochondrial toxicity associated with a metabolic disorder.

Preferably, the metabolic disorders is a drug-induced phospholipidosis or a metabolic disorder caused by inborn errors such as e.g. inborn error of ureagenesis, or an inherited metabolic disorders such as e.g. phenylketonuria. More preferably, the metabolic disorder is drug-induced phospholipidosis.

PAG may be used as marker for determining mitochondrial toxicity in body fluid samples of any animal if said animal has endogenous phenylacetylglycine or an equivalent thereof. Preferably, PAG is used as marker determining mitochondrial toxicity in body fluid samples of human or rodent, whereby the rodent is preferably a rat or a mouse.

The term “biomarker” or “marker” as used herein refers to molecules in an individual which are differentially present (i.e. present in increased or decreased levels) depending on presence or absence of a certain condition, disease, or complication. In particular, biochemical markers are gene expression products which are differentially present (e.g. through increased or decreased level of expression or turnover) in presence or absence of a certain condition, disease, or complication. The level of a suitable biomarker can indicate the presence or absence of a particular condition, disease, or risk, and thus allow diagnosis or determination of the condition, disease or risk.

The present invention also relates to a kit comprising a means or an agent for measuring PAG.

Such a means or agent may be any suitable means or agent known to the person skilled in the art. For example, a suitable agent may be any kind of ligand or antibody specific for measuring said biomarkers. The kit may also comprise any other components deemed appropriate in the context of measuring the level(s) of the respective biomarkers, such as suitable buffers, filters, etc.

Optionally, the kit may additionally comprise a user's manual for interpreting the results of any measurement(s) with respect to determining whether an individual suffers from mitochondrial toxicity associated a metabolic disorder wherein the metabolic disorders is preferably drug-induced phospholipidosis or a metabolic disorder caused by inborn errors such as inborn error of ureagenesis or an inherited metabolic disorders such as e.g. phenylketonuria. Particularly, such manual may include information about what measured level corresponds to an increased level.

The present invention also relates to the use of said kit for assessing mitochondrial toxicity associated with a metabolic disorder in an individual. Furthermore, the invention relates to the use of said kit for determining the risk of a phospholipidotic compound for inducing mitochondrial toxicity which is associated with a metabolic disorder. Preferably, the metabolic disorder is a drug-induced phospholipidosis or a metabolic disorder caused by inborn errors such as e.g. inborn error of ureagenesis, or an inherited metabolic disorder such as e.g. phenylketonuria.

The present invention also relates to the use of said kit in any of the methods according to the present invention for determining the risk of a phospholipidotic compound for inducing mitochondrial toxicity which is associated with a metabolic disorder or for assessing mitochondrial toxicity associated with a metabolic disorder in an individual. Preferably, the metabolic disorder is a drug-induced phospholipidosis or a metabolic disorder caused by inborn errors such as e.g. inborn error of ureagenesis, or an inherited metabolic disorder such as e.g. phenylketonuria.

The term “normal level” as used herein refers to the range of the level of PAG in a body fluid sample of a control. A control is one or more individuals not suffering from mitochondrial toxicity associated with phospholipidosis or the treated animal before treatment (pre-dose values within the same individual). For rats, the number of individuals is preferably higher than 100, more preferably more than 500, most preferably more than 1000. The normal range is determined by methods well known to the skilled person in the art. A preferred method is for example to determine the range of the values between quantile 2.5 and quantile 97.5, which leaves 5% of “normal” values outside the normal range or in other words, it covers 95% of all values of the control.

The pathological status is defined as deviation from the normal status. According to the invention this pathological status is indicated by an increased level of a biomarker. The term “increased level” as used herein refers to the level of PAG in a body fluid sample which is significantly higher than the normal level. Significantly higher means that the level is higher and that the difference to the normal level is statistically relevant (p≦0.05, preferably, p≦0.01).

PAG may also be used as target. Therefore, the present invention provides a method of screening for a compound which interacts with PAG. Such methods are well known in the art.

A suitable method is for example the method of screening for a phospholipidotic compound which interacts with PAG, comprising a) contacting PAG with a compound or a plurality of compounds under conditions which allow interaction of said compound or a plurality of compounds with PAG; and b) detecting the interaction between said compound or plurality of compounds with PAG.

PAG may be immobilized prior step a) or between step a) and step b).

Having now generally described this invention, the same will become better understood by reference to the specific examples, which are included herein for purpose of illustration only and are not intended to be limiting unless otherwise specified, in connection with the following figures.

### FIGURES

FIG. 1 shows the chemical structure of phenylacetylglycine (A); phenylacetylglutamine (B); Compound 1: 2-(3,5-Bis-trifluoromethyl-phenyl)-N-methyl-N-[-(6-(4-methyl-piperazin-1-yl)-4-o-tolyl-pyridin-3-yl]-isobutyramide (C); Compound 2: 2-(3,5-Bis-trifluoromethyl-phenyl)-N-[4-(2-chloro-phenyl)-6-morpholin-4-yl-pyridin-3-yl]-N-methyl-isobutyramide (D).

FIG. 2 shows a graphical representation of a summary of spectral data: The top panel shows one example of a ¹H NMR urine spectrum taken on a control rat. The aromatic region boxed in light grey and the aliphatic region boxed in black contains signals of PAG. This is shown in more detail in the expansion panel (bottom), where 15 spectra of the time point +144 h are shown as a stacked plot.

FIG. 3 shows a graphical representation of the relative mean PAG concentration levels in samples derived from animals treated with compound 2 (2-(3,5-Bis-trifluoromethyl-phenyl)-N-[4-(2-chloro-phenyl)-6-morpholin-4-yl-pyridin-3-yl]-N-methyl-isobutyramide) related to time matched control samples. Control animals were indicated by a square, low-dosed animals (300 mg/kg) by a full circle and high-dosed animals (1000 mg/kg) were depicted in a triangle.

FIG. 4 shows a graphical representation of the relative mean PAG concentration levels of samples derived from animals treated with compound 1 (2-(3,5-Bis-trifluoromethyl-phenyl)-N-methyl-N-[6-(4-methyl-piperazin-l-yl)-4-o-tolyl-pyridin-3-yl]-isobutyramide) related to time matched control samples. Control animals were indicated by a square, low-dosed animals (300 mg/kg) by a full circle and high-dosed animals (1500 mg/kg) were depicted in a triangle. High standard deviations visible for high-dosed animals can be attributed to differences of individual response kinetics and response intensities.

### EXAMPLES

Commercially available reagents referred to in the examples were used according to manufacturer's instructions unless otherwise indicated.

### Example 1

### Animals, Doses and Sampling

Animals: Male Wistar Rats (RCC, Inc., Füllinsdorf)

All animals received (human) care as specified by Swiss law and in accordance with the “Guide for the care and use of laboratory animals” published by the NIH. Male Wistar rats (5 animals/dose-group) were purchased from RCC (Füllingsdorf, Switzerland) and housed individually. Treated animals were dosed orally by gavage with several doses of test compounds (Table 1). Control animals received the same volume of vehicle as placebo. Necropsy was performed 48 (subgroup A) or 168 hours (subgroup B) after a single administration (=Day 1) and urine samples were collected in metabolism cages at 0 to 4° C. (automatically refrigerated by a Tecniplast sampling/cooling unit (RACK B940, Tecniplast, USA)) at the intervals given in the sampling schedule in Table 2 into labelled sample tubes containing 1 ml of an aqueous Na-azide (1%) solution. Before aliquoting, urine volumes were determined.

Compounds:

Compound 1=2-(3,5-Bis-trifluoromethyl-phenyl)-N-methyl-N-[6-(4-methyl-piperazin-1-yl)-4-o-tolyl-pyridin-3-yl]-isobutyramide;

Compound 2=2-(3,5-Bis-trifluoromethyl-phenyl)-N-[4-(2-chloro-phenyl)-6-morpholin-4-yl-pyridin-3-yl]-N-methyl-isobutyramide,

Vehicle/control: Thixtrope vehicle

The structural difference between both molecules can be mainly characterized by the exchange of the piperazine against a morpholine moiety in compound 2. This leads to a down shift of the basic pKa value from 7.67 to 4.07. Besides the reduced amphiphilicity the lower basic pKa value of compound 2 is the most important reason why compound 2 has a low potential and compound 1 a high potential to induce phospholipidosis.

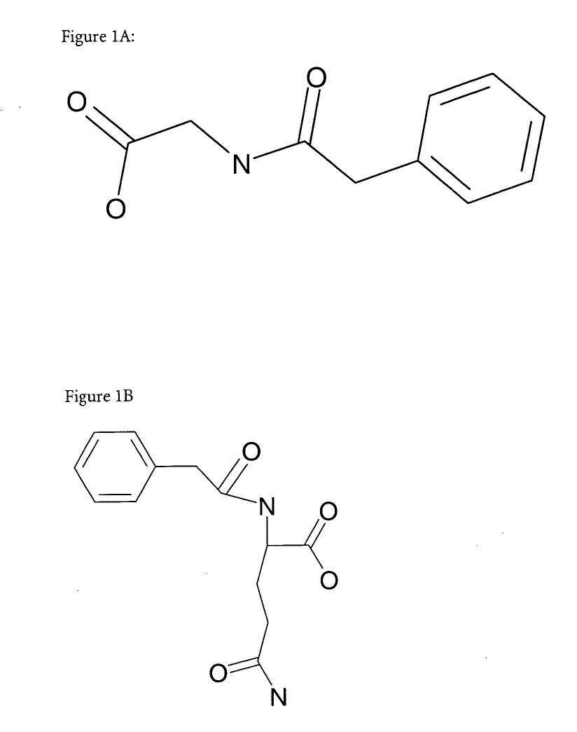

The above described clear differences in the physico-chemical properties of the two compounds allow despite the high structural similarity of both compounds (differences are depicted in red in FIG. 1) a clear distinction of the compounds.

TABLE 1
Group Design.

| | | | Number of rats | Number of rats |
|---|---|---|---|---|
| | | Dose | Subgroup A | Subgroup B |
| | | level | (necropsy 48 | (necropsy 168 |
| Dose | | (mg/kg/ | hours after | hours after |
| Groups | Compound | day) | application) | application) |
| 1 | Vehicle | 0 | 5 (1-5) | 5 (6-10) |
| 2 | Compound 1 | 300 | 5 (11-15) | 5 (16-20) |
| 3 | Compound 1 | 1500 | 5 (21-25) | 5 (26-30) |
| 4 | Compound 2 | 300 | 5 (31-35) | 5 (36-40) |
| 5 | Compound 2 | 1000 | 5 (41-45) | 5 (46-50) |

Compound 1 = 2-(3,5-Bis-trifluoromethyl-phenyl)-N-methyl-N-[6-(4-methyl-piperazin-1-yl)-4-o-tolyl-pyridin-3-yl]-isobutyramide; Compound 2 = 2-(3,5-Bis-trifluoromethyl-phenyl)-N-[4-(2-chloro-phenyl)-6-morpholin-4-yl-pyridin-3-yl]-N-methyl-isobutyramide, Vehicle/control: Thixtrope vehicle

### Example 2

### PAG Measurement by NMR

Urine samples were taken on day -7,-2,-1, 1, 2, 3, 4, 5, 6 and 7, whereby Day 1 was the Day of dosing.

TABLE 2
Urine sampling for analysis.

| | Absolute day | | Groups 1-5 | | |
|---|---|---|---|---|---|
| | of urine sample | | Subgr. | Subgr. | |
| Days | collection | Time | A | B | Label |
| −7 | −7 to −6 (24 h) | Arriv. to −144 h | X | X | −144 h |
| −2 | −2 (0-8 h) | −48 h to −40 h | X | X | −40 h |
| −1 | −1 (0-8 h) | −24 h to −16 h | X | X | −16 h |
| −1 | −1 to 1 (8-24 h) | −16 h to 0 h | X | X | 0 h |
| 1 | 1 (0-8) | 0 h to 8 h | X | X | 8 h |
| 1 | 1 to 2 (8-24 h) | 8 h to 24 h | X | X | 24 h |
| 2 | 2 to 3 (0-24 h) | 24 h to 48 h | X | X | 48 h |
| 3 | 3 to 4 (0-24 h) | 48 h to 72 h | | X | 72 h |
| 4 | 4 to 5 (0-24 h) | 72 h to 96 h | | X | 96 h |
| 5 | 5 to 6 (0-24 h) | 96 h to 120 h | | X | 120 h |
| 6 | 6 to 7 (0-24 h) | 120 h to 144 h | | X | 144 h |
| 7 | 7 to 8 (0-24 h) | 144 h to 168 h | | X | 168 h |

Time = 0 h corresponds to the time of dosing in the morning of day 1

Immediately after reaching the end of the sampling period, the volume was determined and the samples were centrifuged at 3000 u/min (500 g) for 10 minutes.

Aliquot: Approximately 2 ml was transferred in 1.8 ml cryvials, round bottom, freestanding, with screw caps and septum for NMR-spectroscopic examinations (Bruker Analytik GmbH, Rheinstetten, Germany, Bruker part No. 85372). The vials were stored at −80° C.

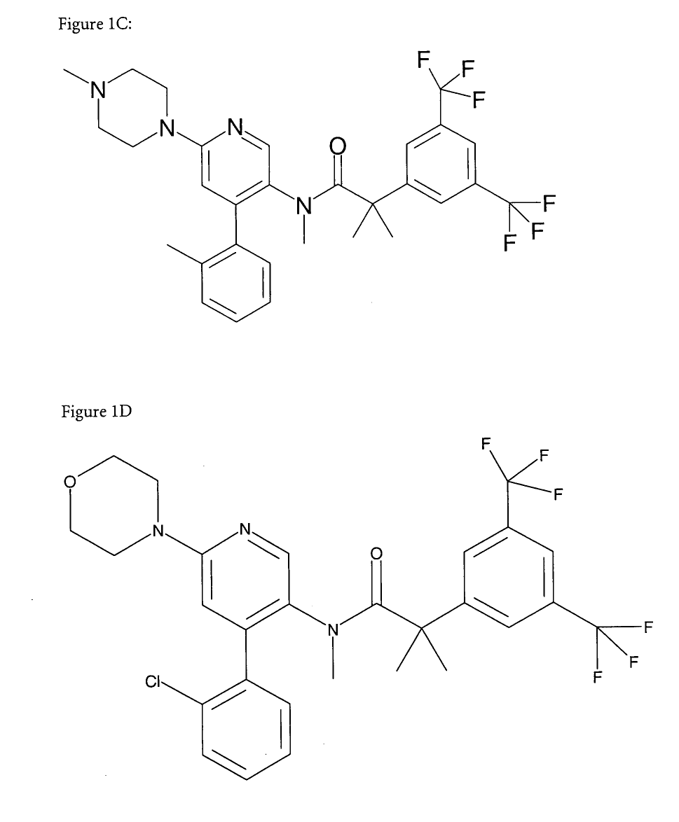

Urine samples were prepared and measured on a Bruker 500 MHz NMR instrument according to the COMET 1H-NMR protocol (Keun, H. C et al., (2002) Physiological variation and analytical reproducibility in metabonomic urinalysis. Chem. Res. Tox. 15, 1380-1386) and as described above. In total 467 urine samples were analyzed. After measurement, all data were processed by XWINMR 3.5.6 (Bruker Biospin AG, Fällanden). Representative spectra are depicted in FIG. 2. Phase correction and baseline correction were performed with NMRPROC 0.3 (T. Ebbels, H. Keun; Imperial College). Spectral data were binned using AMIX 2.6 (Bruker Biospin AG, Fällanden) following the COMET Data Processing & Pattern Recognition protocol (Keun, H. C., et al., (2002) Physiological variation and analytical reproducibility in metabonomic urinalysis. Chem. Res. Tox. 15, 1380-1386). QC check procedures (Dieterle F., Ross A., Schlotterbeck G., Senn H. (2006) Probabilistic Quotient Normalization as Robust Method to Account for Dilution of Complex Biological Mixtures. Application in 1H NMR Metabonomics. Anal. Chem. 78, 4281-4290) were applied on binned data to detect outliers. In total 7 outliers were detected and removed from further analysis. All data were mean-centered and normalized using the probabilistic quotient normalization (Dieterle F., Ross A., Schlotterbeck G., Senn H. (2006) Probabilistic Quotient Normalization as Robust Method to Account for Dilution of Complex Biological Mixtures. Application in 1H NMR Metabonomics. Anal. Chem. 78, 4281-4290.). All binned data were annotated and transferred to the ANT (ANT: Affymetrix NMR Toxicology Data System) database.

The exact PAG quantification was performed according to following procedure:
Baseline subtraction of spectra
Integration of PAG peaks ((=7.47 ppm, (=7.39 ppm, (=3.79 ppm, and (=3.7 ppm respectively) and Citrate region ((=2.66-2.74 ppm and 2.50-2.58 ppm).
Quotient normalization (Dieterle F., Ross A., Schlotterbeck G., Senn H. (2006) Probabilistic Quotient Normalization as Robust Method to Account for Dilution of Complex Biological Mixtures. Application in 1H NMR Metabonomics. Anal. Chem. 78, 4281-4290.)
Divide each of the PAG bins by its average (all spectra) to correct for different relaxation times and number of protons
Calculate median out of the PAG bins
Calculate ratio versus time-matched controls

Compound 2: 2-(3,5-Bis-trifluoromethyl-phenyl)-N-[4-(2-chloro-phenyl)-6-morpholin-4-yl-pyridin-3-yl]-N-methyl-isobutyramide

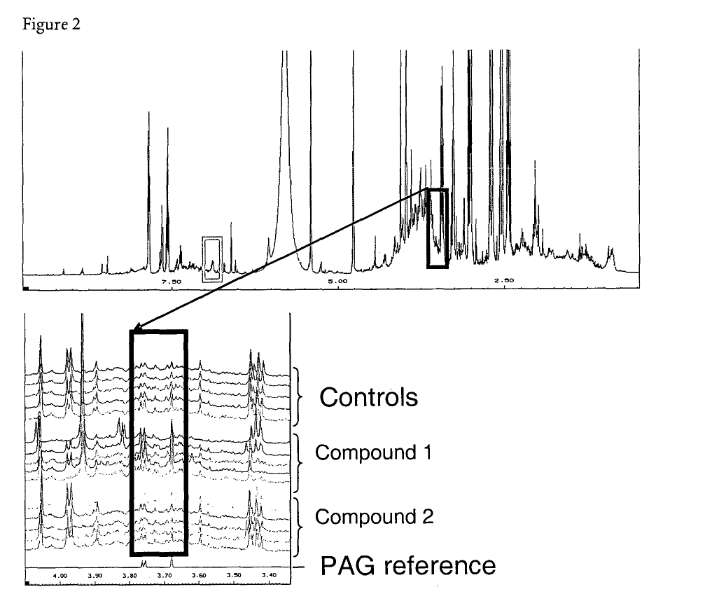

PAG levels were normalized to time matched control animals for compound 2 as described above. No significant dose dependant change of PAG was detected for individual animals dosed with Compound 2 (see FIG. 3 and Table 3). The PAG levels of both dose groups were comparable to their time matched controls.

Compound 1: 2-(3,5-Bis-trifluoromethyl-phenyl)-N-methyl-N-[6-(4-methyl-piperazin-1-yl)-4-o-tolyl-pyridin-3-yl]-isobutyramide

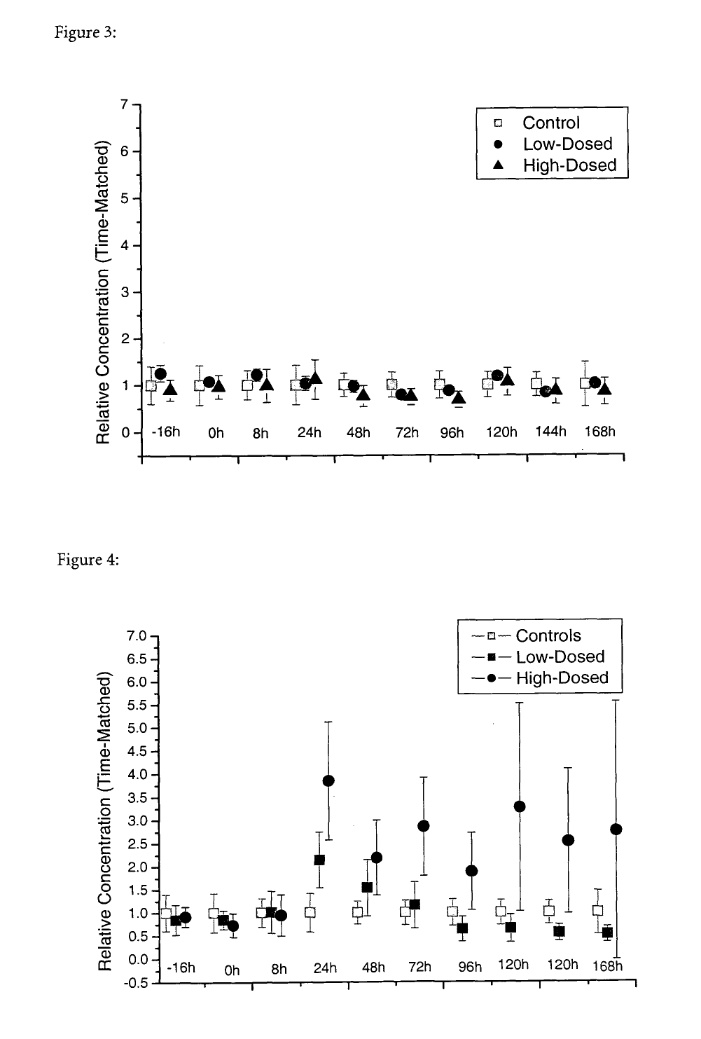

Mean PAG levels were determined (as described above) relatively to time matched controls for all animals dosed with Compound 1. A significant dose dependant elevation of PAG levels was found starting at 24 h after dosing for both dose groups. High-dosed animals show 4-fold increased PAG levels, whereas in low-dosed animals an increase of a factor of two was found compared to time matched controls (see FIG. 4 and Table 3). The levels of low-dosed animals decrease with time and at time points later than 72 h, mean levels of PAG fall below control samples. Mean PAG levels of high-dosed animals remain elevated (3-4 fold increased) until the end of the study.

High standard deviations visible for high-dosed animals can be attributed to differences of individual response kinetics and response intensities.

TABLE 3

| | | | | |
|---|---|---|---|---|
| Spectrum_ID | Group | Animal | Time | PAG |

A: PAG levels in animals 7 days (−114 h) before treatment
with vehicle, compound 1 (2-(3,5-Bis-trifluoromethyl-
phenyl)-N-methyl-N-[6-(4-methyl-piperazin-1-yl)-4-o-tolyl-
pyridin-3-yl]-isobutyramide) or compound 2 (2-(3,5-Bis-
trifluoromethyl-phenyl)-N-[4-(2-chloro-phenyl)-6-morpholin-
4-yl-pyridin-3-yl]-N-methyl-isobutyramide).

| | | | | |
|---|---|---|---|---|
| R118317_10_1 | Control | 1 | −144 | 1.1031 |
| R118318_10_1 | Control | 2 | −144 | 0.7560 |
| R118319_10_1 | Control | 3 | −144 | 0.8519 |
| R118320_10_1 | Control | 4 | −144 | 0.7022 |
| R118321_10_1 | Control | 5 | −144 | 1.1145 |
| R118322_10_1 | Control | 6 | −144 | 0.8381 |
| R118323_10_1 | Control | 7 | −144 | 0.9943 |
| R118324_10_1 | Control | 8 | −144 | 1.2731 |
| R118325_10_1 | Control | 9 | −144 | 0.6968 |
| R118326_10_1 | Control | 10 | −144 | 1.0355 |
| R118327_10_1 | Compound 1_Low_Dose | 11 | −144 | 0.8210 |
| R118328_10_1 | Compound 1_Low_Dose | 12 | −144 | 1.1559 |
| R118329_10_1 | Compound 1_Low_Dose | 13 | −144 | 0.9260 |
| R118330_10_1 | Compound 1_Low_Dose | 14 | −144 | 0.7456 |
| R118331_10_1 | Compound 1_Low_Dose | 15 | −144 | 0.8081 |
| R118332_10_1 | Compound 1_Low_Dose | 16 | −144 | 0.6038 |
| R118333_10_1 | Compound 1_Low_Dose | 17 | −144 | 1.5903 |
| R118334_10_1 | Compound 1_Low_Dose | 18 | −144 | 1.1241 |
| R118335_10_1 | Compound 1_Low_Dose | 19 | −144 | 0.9275 |
| R118336_10_1 | Compound 1_Low_Dose | 20 | −144 | 0.8848 |
| R118337_10_1 | Compound 1_High_Dose | 21 | −144 | 0.7666 |
| R118338_10_1 | Compound 1_High_Dose | 22 | −144 | 0.8823 |
| R118339_10_1 | Compound 1_High_Dose | 23 | −144 | 1.0974 |
| R118340_10_1 | Compound 1_High_Dose | 24 | −144 | 0.6021 |
| R118341_10_1 | Compound 1_High_Dose | 25 | −144 | 1.2709 |
| R118342_10_1 | Compound 1_High_Dose | 26 | −144 | 1.0355 |
| R118343_10_1 | Compound 1_High_Dose | 27 | −144 | 0.6455 |
| R118344_10_1 | Compound 1_High_Dose | 28 | −144 | 0.7716 |
| R118345_10_1 | Compound 1_High_Dose | 29 | −144 | 0.9808 |
| R118346_10_1 | Compound 1_High_Dose | 30 | −144 | 0.7150 |
| R118347_10_1 | Compound 2_Low_Dose | 31 | −144 | 0.8959 |
| R118348_10_1 | Compound 2_Low_Dose | 32 | −144 | 0.9866 |
| R118349_10_1 | Compound 2_Low_Dose | 33 | −144 | 0.6113 |
| R118350_10_1 | Compound 2_Low_Dose | 34 | −144 | 1.0040 |
| R118351_10_1 | Compound 2_Low_Dose | 35 | −144 | 0.8700 |
| R118352_10_1 | Compound 2_Low_Dose | 36 | −144 | 1.0067 |
| R118353_10_1 | Compound 2_Low_Dose | 37 | −144 | 0.8499 |
| R118354_10_1 | Compound 2_Low_Dose | 38 | −144 | 0.9771 |
| R118355_10_1 | Compound 2_Low_Dose | 39 | −144 | 1.1025 |
| R118356_10_1 | Compound 2_Low_Dose | 40 | −144 | 1.5236 |
| R118357_10_1 | Compound 2_High_Dose | 41 | −144 | 1.1113 |
| R118358_10_1 | Compound 2_High_Dose | 42 | −144 | 0.6810 |
| R118359_10_1 | Compound 2_High_Dose | 43 | −144 | 1.0115 |
| R118360_10_1 | Compound 2_High_Dose | 44 | −144 | 0.6066 |
| R118361_10_1 | Compound 2_High_Dose | 45 | −144 | 0.6015 |
| R118362_10_1 | Compound 2_High_Dose | 46 | −144 | 2.0608 |
| R118363_10_1 | Compound 2_High_Dose | 47 | −144 | 2.0477 |
| R118364_10_1 | Compound 2_High_Dose | 48 | −144 | 2.2465 |
| R118365_10_1 | Compound 2_High_Dose | 49 | −144 | 2.5194 |
| R118366_10_1 | Compound 2_High_Dose | 50 | −144 | 2.3206 |

B: PAG levels in animals 2 days (−40 h) before treatment
with vehicle, compound 1 (2-(3,5-Bis-trifluoromethyl-
phenyl)-N-methyl-N-[6-(4-methyl-piperazin-1-yl)-4-o-tolyl-
pyridin-3-yl]-isobutyramide) or compound 2 (2-(3,5-Bis-
trifluoromethyl-phenyl)-N-[4-(2-chloro-phenyl)-6-morpholin-
4-yl-pyridin-3-yl]-N-methyl-isobutyramide).

| | | | | |
|---|---|---|---|---|
| R118368_10_1 | Control | 1 | −40 | 0.4574 |
| R118369_10_1 | Control | 2 | −40 | 0.5685 |
| R118370_10_1 | Control | 3 | −40 | 1.1028 |
| R118371_10_1 | Control | 4 | −40 | 0.2144 |
| R118372_10_1 | Control | 5 | −40 | 0.6290 |
| R118373_10_1 | Control | 6 | −40 | 0.3959 |
| R118374_10_1 | Control | 7 | −40 | 0.4078 |
| R118375_10_1 | Control | 8 | −40 | 0.5413 |
| R118376_10_1 | Control | 9 | −40 | 0.6181 |
| R118377_10_1 | Control | 10 | −40 | 0.7609 |
| R118378_10_1 | Compound 1_Low_Dose | 11 | −40 | 0.2510 |
| R118379_10_1 | Compound 1_Low_Dose | 12 | −40 | 0.3534 |
| R118380_10_1 | Compound 1_Low_Dose | 13 | −40 | 0.5756 |
| R118381_10_1 | Compound 1_Low_Dose | 14 | −40 | 0.4157 |
| R118382_10_1 | Compound 1_Low_Dose | 15 | −40 | 1.0106 |
| R118383_10_1 | Compound 1_Low_Dose | 16 | −40 | 0.3133 |
| R118384_10_1 | Compound 1_Low_Dose | 17 | −40 | 0.6161 |
| R118385_10_1 | Compound 1_Low_Dose | 18 | −40 | 0.5487 |
| R118386_10_1 | Compound 1_Low_Dose | 19 | −40 | 0.7364 |
| R118387_10_1 | Compound 1_Low_Dose | 20 | −40 | 0.2943 |
| R118388_10_1 | Compound 1_High_Dose | 21 | −40 | 0.6913 |
| R118389_10_1 | Compound 1_High_Dose | 22 | −40 | 0.5278 |
| R118390_10_1 | Compound 1_High_Dose | 23 | −40 | 0.8411 |
| R118391_10_1 | Compound 1_High_Dose | 24 | −40 | 0.6109 |
| R118392_10_1 | Compound 1_High_Dose | 25 | −40 | 0.9294 |
| R118393_10_1 | Compound 1_High_Dose | 26 | −40 | 0.6309 |
| R118394_10_1 | Compound 1_High_Dose | 27 | −40 | 0.2161 |
| R118395_10_1 | Compound 1_High_Dose | 28 | −40 | 0.3309 |
| R118396_10_1 | Compound 1_High_Dose | 29 | −40 | 0.3963 |
| R118397_10_1 | Compound 1_High_Dose | 30 | −40 | 0.6621 |
| R118398_10_1 | Compound 2_Low_Dose | 31 | −40 | 1.1566 |
| R118399_10_1 | Compound 2_Low_Dose | 32 | −40 | 1.2483 |
| R118400_10_1 | Compound 2_Low_Dose | 33 | −40 | 0.4473 |
| R118401_10_1 | Compound 2_Low_Dose | 34 | −40 | 1.2604 |
| R118402_10_1 | Compound 2_Low_Dose | 35 | −40 | 0.3418 |
| R118403_10_1 | Compound 2_Low_Dose | 36 | −40 | 0.5238 |
| R118404_10_1 | Compound 2_Low_Dose | 37 | −40 | 0.5336 |
| R118405_10_1 | Compound 2_Low_Dose | 38 | −40 | 0.7115 |
| R118406_10_1 | Compound 2_Low_Dose | 39 | −40 | 0.6105 |
| R118407_10_1 | Compound 2_Low_Dose | 40 | −40 | 0.5232 |
| R118408_10_1 | Compound 2_High_Dose | 41 | −40 | 0.5320 |
| R118409_10_1 | Compound 2_High_Dose | 42 | −40 | 0.2568 |
| R118410_10_1 | Compound 2_High_Dose | 43 | −40 | 0.4805 |
| R118411_10_1 | Compound 2_High_Dose | 44 | −40 | 0.4717 |
| R118412_10_1 | Compound 2_High_Dose | 45 | −40 | 0.4514 |
| R118413_10_1 | Compound 2_High_Dose | 46 | −40 | 0.4760 |
| R118414_10_1 | Compound 2_High_Dose | 47 | −40 | 0.6419 |
| R118415_10_1 | Compound 2_High_Dose | 48 | −40 | 0.7515 |
| R118416_10_1 | Compound 2_High_Dose | 49 | −40 | 0.6794 |
| R118417_10_1 | Compound 2_High_Dose | 50 | −40 | 0.7696 |

C: PAG levels in animals 1 day (−16 h) before treatment
with vehicle, compound 1 (2-(3,5-Bis-trifluoromethyl-
phenyl)-N-methyl-N-[6-(4-methyl-piperazin-1-yl)-4-o-tolyl-
pyridin-3-yl]-isobutyramide) or compound 2 (2-(3,5-Bis-
trifluoromethyl-phenyl)-N-[4-(2-chloro-phenyl)-6-morpholin-
4-yl-pyridin-3-yl]-N-methyl-isobutyramide).

| | | | | |
|---|---|---|---|---|
| R118419_10_1 | Control | 1 | −16 | 0.4757 |
| R118420_10_1 | Control | 2 | −16 | 0.4325 |
| R118421_10_1 | Control | 3 | −16 | 0.5395 |
| R118422_10_1 | Control | 4 | −16 | 0.2286 |
| R118423_10_1 | Control | 5 | −16 | 0.4863 |
| R118424_10_1 | Control | 6 | −16 | 0.5213 |
| R118425_10_1 | Control | 7 | −16 | 0.3688 |
| R118426_10_1 | Control | 8 | −16 | 0.9799 |
| R118427_10_1 | Control | 9 | −16 | 0.8335 |
| R118428_10_1 | Control | 10 | −16 | 0.8029 |
| R118429_10_1 | Compound 1_Low_Dose | 11 | −16 | 0.2393 |
| R118430_10_1 | Compound 1_Low_Dose | 12 | −16 | 0.4349 |
| R118431_10_1 | Compound 1_Low_Dose | 13 | −16 | 0.5528 |
| R118432_10_1 | Compound 1_Low_Dose | 14 | −16 | 0.5649 |
| R118433_10_1 | Compound 1_Low_Dose | 15 | −16 | 0.8171 |
| R118434_10_1 | Compound 1_Low_Dose | 16 | −16 | 0.3201 |
| R118435_10_1 | Compound 1_Low_Dose | 17 | −16 | 0.5236 |
| R118436_10_1 | Compound 1_Low_Dose | 18 | −16 | 0.6045 |
| R118437_10_1 | Compound 1_Low_Dose | 19 | −16 | 0.4646 |
| R118438_10_1 | Compound 1_Low_Dose | 20 | −16 | 0.2537 |
| R118439_10_1 | Compound 1_High_Dose | 21 | −16 | 0.7486 |
| R118440_10_1 | Compound 1_High_Dose | 22 | −16 | 0.5615 |
| R118441_10_1 | Compound 1_High_Dose | 23 | −16 | 0.6169 |
| R118442_10_1 | Compound 1_High_Dose | 24 | −16 | 0.5637 |
| R118443_10_1 | Compound 1_High_Dose | 25 | −16 | 0.4281 |
| R118444_10_1 | Compound 1_High_Dose | 26 | −16 | 0.4828 |
| R118445_10_1 | Compound 1_High_Dose | 27 | −16 | 0.2993 |
| R118446_10_1 | Compound 1_High_Dose | 28 | −16 | 0.4535 |
| R118447_10_1 | Compound 1_High_Dose | 29 | −16 | 0.5361 |
| R118448_10_1 | Compound 1_High_Dose | 30 | −16 | 0.5442 |
| R118449_10_1 | Compound 2_Low_Dose | 31 | −16 | 1.0275 |
| R118450_10_1 | Compound 2_Low_Dose | 32 | −16 | 1.1002 |
| R118451_10_1 | Compound 2_Low_Dose | 33 | −16 | 0.4610 |
| R118452_10_1 | Compound 2_Low_Dose | 34 | −16 | 1.3440 |
| R118453_10_1 | Compound 2_Low_Dose | 35 | −16 | 0.2541 |
| R118454_10_1 | Compound 2_Low_Dose | 36 | −16 | 0.7357 |
| R118455_10_1 | Compound 2_Low_Dose | 37 | −16 | 0.5260 |
| R118456_10_1 | Compound 2_Low_Dose | 38 | −16 | 0.6162 |
| R118457_10_1 | Compound 2_Low_Dose | 39 | −16 | 0.5218 |
| R118458_10_1 | Compound 2_Low_Dose | 40 | −16 | 0.4132 |
| R118459_10_1 | Compound 2_High_Dose | 41 | −16 | 0.6280 |
| R118460_10_1 | Compound 2_High_Dose | 42 | −16 | 0.3499 |
| R118461_10_1 | Compound 2_High_Dose | 43 | −16 | outlier |
| R118462_10_1 | Compound 2_High_Dose | 44 | −16 | 0.4531 |
| R118463_10_1 | Compound 2_High_Dose | 45 | −16 | 0.5078 |
| R118464_10_1 | Compound 2_High_Dose | 46 | −16 | 0.6008 |
| R118465_10_1 | Compound 2_High_Dose | 47 | −16 | 0.3961 |
| R118466_10_1 | Compound 2_High_Dose | 48 | −16 | 0.6035 |
| R118467_10_1 | Compound 2_High_Dose | 49 | −16 | 0.5396 |
| R118468_10_1 | Compound 2_High_Dose | 50 | −16 | 0.6968 |

D: PAG levels in animals within 8 hours (0 h) before treatment
with vehicle, compound 1 (2-(3,5-Bis-trifluoromethyl-
phenyl)-N-methyl-N-[6-(4-methyl-piperazin-1-yl)-4-o-tolyl-
pyridin-3-yl]-isobutyramide) or compound 2 (2-(3,5-Bis-
trifluoromethyl-phenyl)-N-[4-(2-chloro-phenyl)-6-morpholin-
4-yl-pyridin-3-yl]-N-methyl-isobutyramide).

| | | | | |
|---|---|---|---|---|
| R118470_10_1 | Control | 1 | 0 | 0.4509 |
| R118471_10_1 | Control | 2 | 0 | 0.3356 |
| R118472_10_1 | Control | 3 | 0 | 0.3812 |
| R118473_10_1 | Control | 4 | 0 | 0.1276 |
| R118474_10_1 | Control | 5 | 0 | 0.2457 |
| R118475_10_1 | Control | 6 | 0 | 0.3824 |
| R118476_10_1 | Control | 7 | 0 | 0.3107 |
| R118477_10_1 | Control | 8 | 0 | 0.7300 |
| R118478_10_1 | Control | 9 | 0 | 0.5202 |
| R118479_10_1 | Control | 10 | 0 | 0.4288 |
| R118480_10_1 | Compound 1_Low_Dose | 11 | 0 | 0.3142 |
| R118481_10_1 | Compound 1_Low_Dose | 12 | 0 | 0.2440 |
| R118482_10_1 | Compound 1_Low_Dose | 13 | 0 | 0.3422 |
| R118483_10_1 | Compound 1_Low_Dose | 14 | 0 | 0.2530 |
| R118484_10_1 | Compound 1_Low_Dose | 15 | 0 | 0.3700 |
| R118485_10_1 | Compound 1_Low_Dose | 16 | 0 | outlier |
| R118486_10_1 | Compound 1_Low_Dose | 17 | 0 | 0.6319 |
| R118487_10_1 | Compound 1_Low_Dose | 18 | 0 | 0.3708 |
| R118488_10_1 | Compound 1_Low_Dose | 19 | 0 | 0.3307 |
| R118489_10_1 | Compound 1_Low_Dose | 20 | 0 | 0.2336 |
| R118490_10_1 | Compound 1_High_Dose | 21 | 0 | 0.3099 |
| R118491_10_1 | Compound 1_High_Dose | 22 | 0 | 0.4951 |
| R118492_10_1 | Compound 1_High_Dose | 23 | 0 | 0.2661 |
| R118493_10_1 | Compound 1_High_Dose | 24 | 0 | 0.1961 |
| R118494_10_1 | Compound 1_High_Dose | 25 | 0 | 0.1580 |
| R118495_10_1 | Compound 1_High_Dose | 26 | 0 | 0.3419 |
| R118496_10_1 | Compound 1_High_Dose | 27 | 0 | 0.1895 |
| R118497_10_1 | Compound 1_High_Dose | 28 | 0 | 0.2711 |
| R118498_10_1 | Compound 1_High_Dose | 29 | 0 | 0.3305 |
| R118499_10_1 | Compound 1_High_Dose | 30 | 0 | 0.4159 |
| R118500_10_1 | Compound 2_Low_Dose | 31 | 0 | 0.6632 |
| R118501_10_1 | Compound 2_Low_Dose | 32 | 0 | 0.5179 |
| R118502_10_1 | Compound 2_Low_Dose | 33 | 0 | 0.2795 |
| R118503_10_1 | Compound 2_Low_Dose | 34 | 0 | 0.6376 |
| R118504_10_1 | Compound 2_Low_Dose | 35 | 0 | 0.3104 |
| R118505_10_1 | Compound 2_Low_Dose | 36 | 0 | 0.3524 |
| R118506_10_1 | Compound 2_Low_Dose | 37 | 0 | 0.4991 |
| R118507_10_1 | Compound 2_Low_Dose | 38 | 0 | 0.5273 |
| R118508_10_1 | Compound 2_Low_Dose | 39 | 0 | 0.2959 |
| R118509_10_1 | Compound 2_Low_Dose | 40 | 0 | 0.3191 |
| R118510_10_1 | Compound 2_High_Dose | 41 | 0 | 0.3191 |
| R118511_10_1 | Compound 2_High_Dose | 42 | 0 | 0.4082 |
| R118512_10_1 | Compound 2_High_Dose | 43 | 0 | 0.1757 |
| R118513_10_1 | Compound 2_High_Dose | 44 | 0 | 0.4688 |
| R118514_10_1 | Compound 2_High_Dose | 45 | 0 | 0.3904 |
| R118515_10_1 | Compound 2_High_Dose | 46 | 0 | 0.3807 |
| R118516_10_1 | Compound 2_High_Dose | 47 | 0 | 0.4568 |
| R118517_10_1 | Compound 2_High_Dose | 48 | 0 | 0.5015 |
| R118518_10_1 | Compound 2_High_Dose | 49 | 0 | 0.3805 |
| R118519_10_1 | Compound 2_High_Dose | 50 | 0 | 0.4405 |

E: PAG levels in animals within 8 hours (8 h) after treatment
with vehicle, compound 1 (2-(3,5-Bis-trifluoromethyl-
phenyl)-N-methyl-N-[6-(4-methyl-piperazin-1-yl)-4-o-tolyl-
pyridin-3-yl]-isobutyramide) or compound 2 (2-(3,5-Bis-
trifluoromethyl-phenyl)-N-[4-(2-chloro-phenyl)-6-morpholin-
4-yl-pyridin-3-yl]-N-methyl-isobutyramide).

| | | | | |
|---|---|---|---|---|
| R118521_10_1 | Control | 1 | 8 | 0.4611 |
| R118522_10_1 | Control | 2 | 8 | 0.2946 |
| R118523_10_1 | Control | 3 | 8 | 0.6547 |
| R118524_10_1 | Control | 4 | 8 | 0.4422 |
| R118525_10_1 | Control | 5 | 8 | 0.5698 |
| R118526_10_1 | Control | 6 | 8 | 0.8907 |
| R118527_10_1 | Control | 7 | 8 | 0.4853 |
| R118528_10_1 | Control | 8 | 8 | 0.6687 |
| R118529_10_1 | Control | 9 | 8 | 0.5258 |
| R118530_10_1 | Control | 10 | 8 | 0.6474 |
| R118531_10_1 | Compound 1_Low_Dose | 11 | 8 | 1.1423 |
| R118532_10_1 | Compound 1_Low_Dose | 12 | 8 | 0.5270 |
| R118533_10_1 | Compound 1_Low_Dose | 13 | 8 | 0.3918 |
| R118534_10_1 | Compound 1_Low_Dose | 14 | 8 | 0.5331 |
| R118535_10_1 | Compound 1_Low_Dose | 15 | 8 | 0.6815 |
| R118536_10_1 | Compound 1_Low_Dose | 16 | 8 | 0.2977 |
| R118537_10_1 | Compound 1_Low_Dose | 17 | 8 | 0.6756 |
| R118538_10_1 | Compound 1_Low_Dose | 18 | 8 | 0.7527 |
| R118539_10_1 | Compound 1_Low_Dose | 19 | 8 | 0.4111 |
| R118540_10_1 | Compound 1_Low_Dose | 20 | 8 | 0.3688 |
| R118541_10_1 | Compound 1_High_Dose | 21 | 8 | 0.8523 |
| R118542_10_1 | Compound 1_High_Dose | 22 | 8 | 0.4093 |
| R118543_10_1 | Compound 1_High_Dose | 23 | 8 | 0.7986 |
| R118544_10_1 | Compound 1_High_Dose | 24 | 8 | 0.5172 |
| R118545_10_1 | Compound 1_High_Dose | 25 | 8 | 0.2995 |
| R118546_10_1 | Compound 1_High_Dose | 26 | 8 | 0.4361 |
| R118547_10_1 | Compound 1_High_Dose | 27 | 8 | 0.5473 |
| R118548_10_1 | Compound 1_High_Dose | 28 | 8 | 0.4307 |
| R118549_10_1 | Compound 1_High_Dose | 29 | 8 | 1.0174 |
| R118550_10_1 | Compound 1_High_Dose | 30 | 8 | 0.3275 |
| R118551_10_1 | Compound 2_Low_Dose | 31 | 8 | 0.8897 |
| R118552_10_1 | Compound 2_Low_Dose | 32 | 8 | 0.6310 |
| R118553_10_1 | Compound 2_Low_Dose | 33 | 8 | 0.3915 |
| R118554_10_1 | Compound 2_Low_Dose | 34 | 8 | 1.2919 |
| R118555_10_1 | Compound 2_Low_Dose | 35 | 8 | 0.3580 |
| R118556_10_1 | Compound 2_Low_Dose | 36 | 8 | 0.9744 |
| R118557_10_1 | Compound 2_Low_Dose | 37 | 8 | 0.3039 |
| R118558_10_1 | Compound 2_Low_Dose | 38 | 8 | 0.7208 |
| R118559_10_1 | Compound 2_Low_Dose | 39 | 8 | 0.6353 |
| R118560_10_1 | Compound 2_Low_Dose | 40 | 8 | 0.4304 |
| R118561_10_1 | Compound 2_High_Dose | 41 | 8 | 0.6552 |
| R118562_10_1 | Compound 2_High_Dose | 42 | 8 | 0.2654 |
| R118563_10_1 | Compound 2_High_Dose | 43 | 8 | 0.3244 |
| R118564_10_1 | Compound 2_High_Dose | 44 | 8 | 0.3661 |
| R118565_10_1 | Compound 2_High_Dose | 45 | 8 | 0.5090 |
| R118566_10_1 | Compound 2_High_Dose | 46 | 8 | 0.8253 |
| R118567_10_1 | Compound 2_High_Dose | 47 | 8 | 0.6223 |
| R118568_10_1 | Compound 2_High_Dose | 48 | 8 | 0.6794 |
| R118569_10_1 | Compound 2_High_Dose | 49 | 8 | 0.5865 |
| R118570_10_1 | Compound 2_High_Dose | 50 | 8 | 0.5553 |

F: PAG levels in animals within 8 to 24 hours (24 h) after treatment
with vehicle, compound 1 (2-(3,5-Bis-trifluoromethyl-
phenyl)-N-methyl-N-[6-(4-methyl-piperazin-1-yl)-4-o-tolyl-
pyridin-3-yl]-isobutyramide) or compound 2 (2-(3,5-Bis-
trifluoromethyl-phenyl)-N-[4-(2-chloro-phenyl)-6-morpholin-
4-yl-pyridin-3-yl]-N-methyl-isobutyramide).

| | | | | |
|---|---|---|---|---|
| R118572_10_1 | Control | 1 | 24 | 0.3221 |
| R118573_10_1 | Control | 2 | 24 | 0.1270 |
| R118574_10_1 | Control | 3 | 24 | 0.3634 |
| R118575_10_1 | Control | 4 | 24 | 0.1511 |
| R118576_10_1 | Control | 5 | 24 | 0.2565 |
| R118577_10_1 | Control | 6 | 24 | 0.3562 |
| R118578_10_1 | Control | 7 | 24 | 0.2586 |
| R118579_10_1 | Control | 8 | 24 | 0.5263 |
| R118580_10_1 | Control | 9 | 24 | 0.4187 |
| R118581_10_1 | Control | 10 | 24 | 0.4749 |
| R118582_10_1 | Compound 1_Low_Dose | 11 | 24 | outlier |
| R118583_10_1 | Compound 1_Low_Dose | 12 | 24 | outlier |
| R118584_10_1 | Compound 1_Low_Dose | 13 | 24 | outlier |
| R118585_10_1 | Compound 1_Low_Dose | 14 | 24 | 0.6300 |
| R118586_10_1 | Compound 1_Low_Dose | 15 | 24 | outlier |
| R118587_10_1 | Compound 1_Low_Dose | 16 | 24 | 0.8072 |
| R118588_10_1 | Compound 1_Low_Dose | 17 | 24 | 1.0165 |
| R118589_10_1 | Compound 1_Low_Dose | 18 | 24 | 0.7250 |
| R118590_10_1 | Compound 1_Low_Dose | 19 | 24 | 0.5035 |
| R118591_10_1 | Compound 1_Low_Dose | 20 | 24 | 0.6637 |
| R118592_10_1 | Compound 1_High_Dose | 21 | 24 | 1.7579 |
| R118593_10_1 | Compound 1_High_Dose | 22 | 24 | 1.3035 |
| R118594_10_1 | Compound 1_High_Dose | 23 | 24 | 1.4190 |
| R118595_10_1 | Compound 1_High_Dose | 24 | 24 | 0.4001 |
| R118596_10_1 | Compound 1_High_Dose | 25 | 24 | 0.8788 |
| R118597_10_1 | Compound 1_High_Dose | 26 | 24 | 1.2898 |
| R118598_10_1 | Compound 1_High_Dose | 27 | 24 | 1.4210 |
| R118599_10_1 | Compound 1_High_Dose | 28 | 24 | 1.9488 |
| R118600_10_1 | Compound 1_High_Dose | 29 | 24 | 1.6305 |
| R118601_10_1 | Compound 1_High_Dose | 30 | 24 | 1.2496 |
| R118602_10_1 | Compound 2_Low_Dose | 31 | 24 | 0.6934 |
| R118603_10_1 | Compound 2_Low_Dose | 32 | 24 | 0.3762 |
| R118604_10_1 | Compound 2_Low_Dose | 33 | 24 | 0.4835 |
| R118605_10_1 | Compound 2_Low_Dose | 34 | 24 | 0.8446 |
| R118606_10_1 | Compound 2_Low_Dose | 35 | 24 | 0.3743 |
| R118607_10_1 | Compound 2_Low_Dose | 36 | 24 | 0.5615 |
| R118608_10_1 | Compound 2_Low_Dose | 37 | 24 | 0.3777 |
| R118609_10_1 | Compound 2_Low_Dose | 38 | 24 | 0.4892 |
| R118610_10_1 | Compound 2_Low_Dose | 39 | 24 | 0.3393 |
| R118611_10_1 | Compound 2_Low_Dose | 40 | 24 | 0.4166 |
| R118612_10_1 | Compound 2_High_Dose | 41 | 24 | 0.4048 |
| R118613_10_1 | Compound 2_High_Dose | 42 | 24 | 0.3363 |
| R118614_10_1 | Compound 2_High_Dose | 43 | 24 | 0.1408 |
| R118615_10_1 | Compound 2_High_Dose | 44 | 24 | 0.3750 |
| R118616_10_1 | Compound 2_High_Dose | 45 | 24 | 0.4284 |
| R118617_10_1 | Compound 2_High_Dose | 46 | 24 | 0.6538 |
| R118618_10_1 | Compound 2_High_Dose | 47 | 24 | 0.4408 |
| R118619_10_1 | Compound 2_High_Dose | 48 | 24 | 0.2588 |
| R118620_10_1 | Compound 2_High_Dose | 49 | 24 | 0.4111 |
| R118621_10_1 | Compound 2_High_Dose | 50 | 24 | 0.3592 |

G: PAG levels in animals 2 days (48 h) after treatment with vehicle,
compound 1 (2-(3,5-Bis-trifluoromethyl-phenyl)-N-methyl-N-[6-(4-
methyl-piperazin-1-yl)-4-o-tolyl-pyridin-3-yl]-isobutyramide)
or compound 2 (2-(3,5-Bis-trifluoromethyl-phenyl)-N-[4-(2-chloro-
phenyl)-6-morpholin-4-yl-pyridin-3-yl]-N-methyl-isobutyramide).

| | | | | |
|---|---|---|---|---|
| R118623_10_1 | Control | 1 | 48 | 0.3650 |
| R118625_10_1 | Control | 3 | 48 | 0.5825 |
| R118626_10_1 | Control | 4 | 48 | 0.2236 |
| R118627_10_1 | Control | 5 | 48 | 0.3294 |
| R118628_10_1 | Control | 6 | 48 | 0.4203 |
| R118629_10_1 | Control | 7 | 48 | 0.4495 |
| R118630_10_1 | Control | 8 | 48 | 0.3544 |
| R118631_10_1 | Control | 9 | 48 | 0.4253 |
| R118632_10_1 | Control | 10 | 48 | 0.4665 |
| R118633_10_1 | Compound 1_Low_Dose | 11 | 48 | 0.7584 |
| R118634_10_1 | Compound 1_Low_Dose | 12 | 48 | 0.6368 |
| R118635_10_1 | Compound 1_Low_Dose | 13 | 48 | 0.9878 |
| R118636_10_1 | Compound 1_Low_Dose | 14 | 48 | 0.6461 |
| R118637_10_1 | Compound 1_Low_Dose | 15 | 48 | 1.0386 |
| R118638_10_1 | Compound 1_Low_Dose | 16 | 48 | 0.3452 |
| R118639_10_1 | Compound 1_Low_Dose | 17 | 48 | 0.5698 |
| R118640_10_1 | Compound 1_Low_Dose | 18 | 48 | 0.3492 |
| R118641_10_1 | Compound 1_Low_Dose | 19 | 48 | 0.6477 |
| R118642_10_1 | Compound 1_Low_Dose | 20 | 48 | 0.4450 |
| R118643_10_1 | Compound 1_High_Dose | 21 | 48 | 1.0283 |
| R118644_10_1 | Compound 1_High_Dose | 22 | 48 | 0.8835 |
| R118645_10_1 | Compound 1_High_Dose | 23 | 48 | 0.8293 |
| R118646_10_1 | Compound 1_High_Dose | 24 | 48 | 0.9716 |
| R118647_10_1 | Compound 1_High_Dose | 25 | 48 | 0.7369 |
| R118648_10_1 | Compound 1_High_Dose | 26 | 48 | 0.8250 |
| R118649_10_1 | Compound 1_High_Dose | 27 | 48 | 1.8515 |
| R118650_10_1 | Compound 1_High_Dose | 28 | 48 | 0.8422 |
| R118651_10_1 | Compound 1_High_Dose | 29 | 48 | 0.5643 |
| R118652_10_1 | Compound 1_High_Dose | 30 | 48 | 0.5655 |
| R118653_10_1 | Compound 2_Low_Dose | 31 | 48 | 0.6453 |
| R118654_10_1 | Compound 2_Low_Dose | 32 | 48 | 0.3298 |
| R118655_10_1 | Compound 2_Low_Dose | 33 | 48 | 0.3762 |
| R118656_10_1 | Compound 2_Low_Dose | 34 | 48 | 0.7958 |
| R118657_10_1 | Compound 2_Low_Dose | 35 | 48 | 0.1879 |
| R118658_10_1 | Compound 2_Low_Dose | 36 | 48 | 0.5409 |
| R118659_10_1 | Compound 2_Low_Dose | 37 | 48 | 0.2716 |
| R118660_10_1 | Compound 2_Low_Dose | 38 | 48 | 0.3303 |
| R118661_10_1 | Compound 2_Low_Dose | 39 | 48 | 0.3239 |
| R118662_10_1 | Compound 2_Low_Dose | 40 | 48 | 0.3237 |
| R118663_10_1 | Compound 2_High_Dose | 41 | 48 | 0.2358 |
| R118664_10_1 | Compound 2_High_Dose | 42 | 48 | 0.2482 |
| R118665_10_1 | Compound 2_High_Dose | 43 | 48 | 0.3576 |
| R118666_10_1 | Compound 2_High_Dose | 44 | 48 | 0.3535 |
| R118667_10_1 | Compound 2_High_Dose | 45 | 48 | 0.4627 |
| R118668_10_1 | Compound 2_High_Dose | 46 | 48 | 0.4539 |
| R118669_10_1 | Compound 2_High_Dose | 47 | 48 | 0.2073 |
| R118670_10_1 | Compound 2_High_Dose | 48 | 48 | 0.2888 |
| R118671_10_1 | Compound 2_High_Dose | 49 | 48 | 0.2743 |
| R118672_10_1 | Compound 2_High_Dose | 50 | 48 | 0.3092 |

H: PAG levels in animals 3 days (72 h) after treatment with vehicle,
compound 1 (2-(3,5-Bis-trifluoromethyl-phenyl)-N-methyl-N-[6-(4-
methyl-piperazin-1-yl)-4-o-tolyl-pyridin-3-yl]-isobutyramide)
or compound 2 (2-(3,5-Bis-trifluoromethyl-phenyl)-N-[4-(2-chloro-
phenyl)-6-morpholin-4-yl-pyridin-3-yl]-N-methyl-isobutyramide).

| | | | | |
|---|---|---|---|---|
| R118674_10_1 | Control | 6 | 72 | 0.3258 |
| R118675_10_1 | Control | 7 | 72 | 0.2822 |
| R118676_10_1 | Control | 8 | 72 | 0.6112 |
| R118677_10_1 | Control | 9 | 72 | 0.4703 |
| R118678_10_1 | Control | 10 | 72 | 0.4345 |
| R118679_10_1 | Compound 1_Low_Dose | 16 | 72 | 0.3674 |
| R118680_10_1 | Compound 1_Low_Dose | 17 | 72 | 0.3499 |
| R118681_10_1 | Compound 1_Low_Dose | 18 | 72 | 0.6478 |
| R118682_10_1 | Compound 1_Low_Dose | 19 | 72 | 0.7468 |
| R118683_10_1 | Compound 1_Low_Dose | 20 | 72 | 0.3577 |
| R118684_10_1 | Compound 1_High_Dose | 26 | 72 | 1.9720 |
| R118685_10_1 | Compound 1_High_Dose | 27 | 72 | 1.1100 |
| R118686_10_1 | Compound 1_High_Dose | 28 | 72 | 0.7959 |
| R118687_10_1 | Compound 1_High_Dose | 29 | 72 | 0.8368 |
| R118688_10_1 | Compound 1_High_Dose | 30 | 72 | 1.5672 |
| R118689_10_1 | Compound 2_Low_Dose | 36 | 72 | 0.3432 |
| R118690_10_1 | Compound 2_Low_Dose | 37 | 72 | 0.2797 |
| R118691_10_1 | Compound 2_Low_Dose | 38 | 72 | 0.3031 |
| R118692_10_1 | Compound 2_Low_Dose | 39 | 72 | 0.3459 |
| R118693_10_1 | Compound 2_Low_Dose | 40 | 72 | 0.3244 |
| R118694_10_1 | Compound 2_High_Dose | 46 | 72 | 0.3128 |
| R118695_10_1 | Compound 2_High_Dose | 47 | 72 | 0.3653 |
| R118696_10_1 | Compound 2_High_Dose | 48 | 72 | 0.2427 |
| R118697_10_1 | Compound 2_High_Dose | 49 | 72 | 0.2488 |
| R118698_10_1 | Compound 2_High_Dose | 50 | 72 | 0.3522 |

I: PAG levels in animals 4 days (96 h) after treatment with vehicle,
compound 1 (2-(3,5-Bis-trifluoromethyl-phenyl)-N-methyl-N-[6-(4-
methyl-piperazin-1-yl)-4-o-tolyl-pyridin-3-yl]-isobutyramide)
or compound 2 (2-(3,5-Bis-trifluoromethyl-phenyl)-N-[4-(2-chloro-
phenyl)-6-morpholin-4-yl-pyridin-3-yl]-N-methyl-isobutyramide).

| | | | | |
|---|---|---|---|---|
| R118700_10_1 | Control | 6 | 96 | 0.3697 |
| R118701_10_1 | Control | 7 | 96 | 0.3249 |
| R118702_10_1 | Control | 8 | 96 | 0.3650 |
| R118703_10_1 | Control | 9 | 96 | 0.6536 |
| R118704_10_1 | Control | 10 | 96 | 0.3976 |
| R118705_10_1 | Compound 1_Low_Dose | 16 | 96 | 0.3038 |
| R118706_10_1 | Compound 1_Low_Dose | 17 | 96 | 0.1334 |
| R118707_10_1 | Compound 1_Low_Dose | 18 | 96 | 0.2845 |
| R118708_10_1 | Compound 1_Low_Dose | 19 | 96 | 0.4082 |
| R118709_10_1 | Compound 1_Low_Dose | 20 | 96 | 0.2536 |
| R118710_10_1 | Compound 1_High_Dose | 26 | 96 | 0.4112 |
| R118711_10_1 | Compound 1_High_Dose | 27 | 96 | 1.2414 |
| R118712_10_1 | Compound 1_High_Dose | 28 | 96 | 0.7087 |
| R118713_10_1 | Compound 1_High_Dose | 29 | 96 | 1.1422 |
| R118714_10_1 | Compound 1_High_Dose | 30 | 96 | 0.5451 |
| R118715_10_1 | Compound 2_Low_Dose | 36 | 96 | 0.4553 |
| R118716_10_1 | Compound 2_Low_Dose | 37 | 96 | 0.3382 |
| R118717_10_1 | Compound 2_Low_Dose | 38 | 96 | 0.3112 |
| R118718_10_1 | Compound 2_Low_Dose | 39 | 96 | 0.3610 |
| R118719_10_1 | Compound 2_Low_Dose | 40 | 96 | 0.3329 |
| R118720_10_1 | Compound 2_High_Dose | 46 | 96 | 0.3558 |
| R118721_10_1 | Compound 2_High_Dose | 47 | 96 | 0.3303 |
| R118722_10_1 | Compound 2_High_Dose | 48 | 96 | 0.1808 |
| R118723_10_1 | Compound 2_High_Dose | 49 | 96 | 0.2551 |
| R118724_10_1 | Compound 2_High_Dose | 50 | 96 | 0.2381 |

J: PAG levels in animals 5 days (120 h) after treatment with vehicle,
compound 1 (2-(3,5-Bis-trifluoromethyl-phenyl)-N-methyl-N-[6-(4-
methyl-piperazin-1-yl)-4-o-tolyl-pyridin-3-yl]-isobutyramide)
or compound 2 (2-(3,5-Bis-trifluoromethyl-phenyl)-N-[4-(2-chloro-
phenyl)-6-morpholin-4-yl-pyridin-3-yl]-N-methyl-isobutyramide).

| | | | | |
|---|---|---|---|---|
| R118726_10_1 | Control | 6 | 120 | 0.3323 |
| R118727_10_1 | Control | 7 | 120 | 0.2540 |
| R118728_10_1 | Control | 8 | 120 | 0.2119 |
| R118729_10_1 | Control | 9 | 120 | 0.4552 |
| R118730_10_1 | Control | 10 | 120 | 0.4387 |
| R118731_10_1 | Compound 1_Low_Dose | 16 | 120 | 0.2511 |
| R118732_10_1 | Compound 1_Low_Dose | 17 | 120 | 0.1429 |
| R118733_10_1 | Compound 1_Low_Dose | 18 | 120 | 0.1575 |
| R118734_10_1 | Compound 1_Low_Dose | 19 | 120 | 0.2294 |
| R118735_10_1 | Compound 1_Low_Dose | 20 | 120 | 0.3450 |
| R118736_10_1 | Compound 1_High_Dose | 26 | 120 | 0.6995 |
| R118737_10_1 | Compound 1_High_Dose | 27 | 120 | 1.2457 |
| R118738_10_1 | Compound 1_High_Dose | 28 | 120 | 0.6107 |
| R118739_10_1 | Compound 1_High_Dose | 29 | 120 | 2.4430 |
| R118740_10_1 | Compound 1_High_Dose | 30 | 120 | 0.7116 |
| R118741_10_1 | Compound 2_Low_Dose | 36 | 120 | 0.5135 |
| R118742_10_1 | Compound 2_Low_Dose | 37 | 120 | 0.2422 |
| R118743_10_1 | Compound 2_Low_Dose | 38 | 120 | 0.5300 |
| R118744_10_1 | Compound 2_Low_Dose | 39 | 120 | 0.4505 |
| R118745_10_1 | Compound 2_Low_Dose | 40 | 120 | 0.3250 |
| R118746_10_1 | Compound 2_High_Dose | 46 | 120 | 0.4512 |
| R118747_10_1 | Compound 2_High_Dose | 47 | 120 | 0.3983 |
| R118748_10_1 | Compound 2_High_Dose | 48 | 120 | 0.1947 |
| R118749_10_1 | Compound 2_High_Dose | 49 | 120 | 0.3433 |
| R118750_10_1 | Compound 2_High_Dose | 50 | 120 | 0.3880 |

K: PAG levels in animals 6 days (144 h) after treatment with vehicle,
compound 1 (2-(3,5-Bis-trifluoromethyl-phenyl)-N-methyl-N-[6-(4-
methyl-piperazin-1-yl)-4-o-tolyl-pyridin-3-yl]-isobutyramide)
or compound 2 (2-(3,5-Bis-trifluoromethyl-phenyl)-N-[4-(2-chloro-
phenyl)-6-morpholin-4-yl-pyridin-3-yl]-N-methyl-isobutyramide).

| | | | | |
|---|---|---|---|---|
| R118752_10_1 | Control | 6 | 144 | 0.3102 |
| R118753_10_1 | Control | 7 | 144 | 0.3237 |
| R118754_10_1 | Control | 8 | 144 | 0.2400 |
| R118755_10_1 | Control | 9 | 144 | 0.4053 |
| R118756_10_1 | Control | 10 | 144 | 0.5319 |
| R118757_10_1 | Compound 1_Low_Dose | 16 | 144 | 0.2149 |
| R118758_10_1 | Compound 1_Low_Dose | 17 | 144 | 0.1279 |
| R118759_10_1 | Compound 1_Low_Dose | 18 | 144 | 0.2475 |
| R118760_10_1 | Compound 1_Low_Dose | 19 | 144 | 0.2412 |
| R118761_10_1 | Compound 1_Low_Dose | 20 | 144 | 0.2688 |
| R118762_10_1 | Compound 1_High_Dose | 26 | 144 | outlier |
| R118763_10_1 | Compound 1_High_Dose | 27 | 144 | 0.9646 |
| R118764_10_1 | Compound 1_High_Dose | 28 | 144 | 0.3602 |
| R118765_10_1 | Compound 1_High_Dose | 29 | 144 | 1.7596 |
| R118766_10_1 | Compound 1_High_Dose | 30 | 144 | 0.8435 |
| R118767_10_1 | Compound 2_Low_Dose | 36 | 144 | 0.2186 |
| R118768_10_1 | Compound 2_Low_Dose | 37 | 144 | 0.2282 |
| R118769_10_1 | Compound 2_Low_Dose | 38 | 144 | 0.3879 |
| R118770_10_1 | Compound 2_Low_Dose | 39 | 144 | 0.4435 |
| R118771_10_1 | Compound 2_Low_Dose | 40 | 144 | 0.3302 |
| R118772_10_1 | Compound 2_High_Dose | 46 | 144 | 0.4526 |
| R118773_10_1 | Compound 2_High_Dose | 47 | 144 | 0.3196 |
| R118774_10_1 | Compound 2_High_Dose | 48 | 144 | 0.2000 |
| R118775_10_1 | Compound 2_High_Dose | 49 | 144 | 0.2590 |
| R118776_10_1 | Compound 2_High_Dose | 50 | 144 | 0.3791 |

L: PAG levels in animals 7 days (168 h) after treatment with vehicle,
compound 1 (2-(3,5-Bis-trifluoromethyl-phenyl)-N-methyl-N-[6-(4-
methyl-piperazin-1-yl)-4-o-tolyl-pyridin-3-yl]-isobutyramide)
or compound 2 (2-(3,5-Bis-trifluoromethyl-phenyl)-N-[4-(2-chloro-
phenyl)-6-morpholin-4-yl-pyridin-3-yl]-N-methyl-isobutyramide).

| | | | | |
|---|---|---|---|---|
| R118778_10_1 | Control | 6 | 168 | 0.2738 |
| R118779_10_1 | Control | 7 | 168 | 0.1810 |
| R118780_10_1 | Control | 8 | 168 | 0.1795 |
| R118781_10_1 | Control | 9 | 168 | 0.3639 |
| R118782_10_1 | Control | 10 | 168 | 0.4539 |
| R118783_10_1 | Compound 1_Low_Dose | 16 | 168 | 0.1903 |
| R118784_10_1 | Compound 1_Low_Dose | 17 | 168 | 0.1053 |
| R118785_10_1 | Compound 1_Low_Dose | 18 | 168 | 0.1619 |
| R118786_10_1 | Compound 1_Low_Dose | 19 | 168 | 0.1736 |
| R118787_10_1 | Compound 1_Low_Dose | 20 | 168 | 0.2033 |
| R118789_10_1 | Compound 1_High_Dose | 27 | 168 | 0.2991 |
| R118790_10_1 | Compound 1_High_Dose | 28 | 168 | 0.1745 |
| R118791_10_1 | Compound 1_High_Dose | 29 | 168 | 1.9217 |
| R118792_10_1 | Compound 1_High_Dose | 30 | 168 | 0.6983 |
| R118793_10_1 | Compound 2_Low_Dose | 36 | 168 | 0.2454 |
| R118794_10_1 | Compound 2_Low_Dose | 37 | 168 | 0.2715 |
| R118795_10_1 | Compound 2_Low_Dose | 38 | 168 | 0.3498 |
| R118796_10_1 | Compound 2_Low_Dose | 39 | 168 | 0.3157 |
| R118797_10_1 | Compound 2_Low_Dose | 40 | 168 | 0.3238 |
| R118798_10_1 | Compound 2_High_Dose | 46 | 168 | 0.3469 |
| R118799_10_1 | Compound 2_High_Dose | 47 | 168 | 0.2856 |
| R118800_10_1 | Compound 2_High_Dose | 48 | 168 | 0.1695 |
| R118801_10_1 | Compound 2_High_Dose | 49 | 168 | 0.2008 |
| R118802_10_1 | Compound 2_High_Dose | 50 | 168 | 0.2243 |

### Example 3

### Electron Microscopic Examination (Determination of Phospholipidosis)

Blood samples (as much as possible) were withdrawn from all animals at necropsy at about 48 hours after dosing (subgroup A) and about 168 hours after dosing (subgroup B)

The samples were collected from the abdominal aorta or by cardiac puncture under terminal anaesthesia (CO₂). A total of at least 2 ml blood was required to gain buffy coat (buffy coat=leukocyte band) samples.

After fixation in 2.5% glutaraldehyde, the buffy coat samples from all animals were embedded in Epon. Semithin and thin sections were prepared from samples of all vehicle, all compound 1 treated and compound 2 high dose treated animals (animals treated with the lower dose of compound 2 were not examined as the high dose animals did not show any lymphocytes with lamellar bodies). All thin sections were examined ultrastructurally. If possible, 200 lymphocytes per sample were examined for lamellar bodies. The results per sample include the total number of lymphocytes examined, the total number of positive lymphocytes (i.e. with lamellar bodies), the percentage of positive lymphocytes and a grading of severity of phospholipidosis (criteria see table 4)

Results

Lymphocytes containing cytoplasmic lamellar bodies were seen in animals treated with compound 1 only. Animals treated with compound 2 were not affected.

Lamellar bodies occurred dose dependently in animals treated with compound 1. The incidence of affected lymphocytes per animal was quite variable within the treatment groups.

In animals given 300 mg/kg/day of compound 1 and necropsied 48 hours after application the incidence of affected lymphocytes ranged from 5-12% (data from 3 animals). Phospholipidosis was considered to be minimal to slight. At the same dose and necropsy 168 hours after application 0-1% of the lymphocytes were affected only indicating partial or complete recovery. Phospholipidosis was considered to be minimal or not present at this time point.

In animals given 1500 mg/kg/day compound 1 and necropsied 48 hours after application the incidence of affected lymphocytes ranged from 15-31% (data from 4 animals) and phospholipidosis was considered to be moderate to marked. In animals sacrificed 168 hours after application the incidence of affected lymphocytes ranged from 21-31% (data from 4 animals) and phospholipidosis was again considered to be moderate to marked. There was a minimally increased mean incidence in affected lymphocytes in animals sacrificed 168 hours after application when compared to animals sacrificed 48 hours after application. However, this increase was considered to be due to individual variations within the small sample size (4 animals only) rather than a real effect.

Discussion and Conclusion

Lymphocytes containing cytoplasmic lamellar bodies indicating a compound-induced phospholipidosis were seen in animals treated with compound 1 only. Lamellar bodies occurred dose dependently and were already seen 48 hours after application. At 300 mg/kg/day of compound 1 the incidence of affected lymphocytes was about 5-12 ° A) and partial or complete recovery was seen 168 hours after application. At 1500 mg/kg/day of compound 1 15-31% of the lymphocytes were affected. There was no obvious difference in the incidence of affected lymphocytes between the two different time points, i.e. there were no indications of recovery within 168 hours after application. Animals treated with compound 2 did not show any lymphocytes containing cytoplasmic lamellar bodies.

TABLE 4
Individual Animal Data for phospholipidosis.

| | | | No. of animal: | | | | | | |
|---|---|---|---|---|---|---|---|---|---|
| | | | Necropsy after | | | | | | |
| | | | application | | | | | | |
| Dose | | Dose | 48 | 168 | lymphocytes | | | Result, PL | |
| Group | Compound | [mg/kg/day] | hours | hours | counted | positive | %, pos. | − | + |
| 1 | Vehicle | 0 | 1 | | 103 | 0 | 0 | − | |
| | | | 2 | | 90 | 0 | 0 | − | |
| | | | 3 | | 182 | 0 | 0 | − | |
| | | | 4 | | 167 | 0 | 0 | − | |
| | | | 5 | | 109 | 0 | 0 | − | |
| | | | | 6 | 178 | 0 | 0 | − | |
| | | | | 7 | 200 | 0 | 0 | − | |
| | | | | 8 | 133 | 0 | 0 | − | |
| | | | | 9 | 200 | 0 | 0 | − | |
| | | | | 10 | 190 | 0 | 0 | − | |
| 2 | Compound 1 | 300 | 11 | | n.d. ¹ | n.d. ¹ | n.d. ¹ | | n.d. |
| | | | 12 | | 200 | 10 | 5 | | 1 |
| | | | 13 | | n.d. ¹ | n.d. ¹ | n.d. ¹ | | n.d. |
| | | | 14 | | 141 | 8 | 5.7 | | 2 |
| | | | 15 | | 189 | 23 | 12.2 | | 2 |
| | | | | 16 | 82 | 0 | 0 | | − |
| | | | | 17 | 174 | 2 | 1.1 | | 1 |
| | | | | 18 | 200 | 2 | 1 | | 1 |
| | | | | 19 | 200 | 0 | 0 | | − |
| | | | | 20 | 118 | 1 | 0.8 | | 1 |
| 3 | Compound 1 | 1500 | 21 | | 191 | 36 | 18.8 | | 3 |
| | | | 22 | | 126 | 19 | 15.1 | | 3 |
| | | | 23 | | 200 | 61 | 31 | | 4 |
| | | | 24 | | (52) ² | (3) | (5.8) | | (2) |
| | | | 25 | | 113 | 29 | 25.7 | | 4 |
| | | | | 26 | n.d. ⁴ | n.d. ⁴ | n.d. ⁴ | | n.d. |
| | | | | 27 | 200 | 62 | 31 | | 4 |
| | | | | 28 | 200 | 42 | 21 | | 3 |
| | | | | 29 | 103 | 29 | 28.2 | | 4 |
| | | | | 30 | 104 | 25 | 24 | | 3 |
| 4 | Compound 2 | 300 | 31 | | n.d. ³ | n.d. ³ | n.d. ³ | | n.d. |
| | | | 32 | | n.d. ³ | n.d. ³ | n.d. ³ | | n.d. |
| | | | 33 | | n.d. ³ | n.d. ³ | n.d. ³ | | n.d. |
| | | | 34 | | n.d. ³ | n.d. ³ | n.d. ³ | | n.d. |
| | | | 35 | | .n.d. ³ | .n.d. ³ | .n.d. ³ | | n.d. |
| | | | | 36 | n.d. ³ | n.d. ³ | n.d. ³ | | n.d. |
| | | | | 37 | n.d. ³ | n.d. ³ | n.d. ³ | | n.d. |
| | | | | 38 | n.d. ³ | n.d. ³ | n.d. ³ | | n.d. |
| | | | | 39 | n.d. ³ | n.d. ³ | n.d. ³ | | n.d. |
| | | | | 40 | n.d. ³ | n.d. ³ | n.d. ³ | | n.d. |
| 5 | Compound 2 | 1000 | 41 | | 181 | 0 | 0 | − | |
| | | | 42 | | 162 | 0 | 0 | − | |
| | | | 43 | | 200 | 0 | 0 | − | |
| | | | 44 | | 200 | 0 | 0 | − | |
| | | | 45 | | 200 | 0 | 0 | − | |
| | | | | 46 | 200 | 0 | 0 | − | |
| | | | | 47 | 190 | 0 | 0 | − | |
| | | | | 48 | 106 | 0 | 0 | − | |
| | | | | 49 | 173 | 0 | 0 | − | |
| | | | | 50 | 200 | 0 | 0 | − | |

n.d. = not determined;
¹ = insufficient number of lymphocytes;
² = very limited number of lymphocytes (data not included in final assessment);
³ = no findings in higher dose group;
⁴ = no buffy coat available (decedent)
Buffy coats, grade PL (severity of phospholipidosis): ≦5%: minimal, >5-15%: slight, >15-25%: moderate, >25: marked.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. A method for determining risk of inducing mitochondrial toxicity associated with metabolic disorder via treatment of a subject with a phospholipidotic compound, said method comprising
a) obtaining a body fluid sample from a suspect treated with a phospholipidotic compound, wherein the body fluid sample is blood or urine
b) measuring and determining the level of PAG in the body fluid sample, wherein the PAG is phenylacetylglycine, phenylacetylglutamine or equivalents thereof,
c) comparing the level of the determined PAG in the body fluid sample with the level of PAG in a control, wherein an increased level of PAG in the body fluid sample in comparison to the control is indicative that said phospholipidotic compound induces mitochondrial toxicity associated with metabolic disorder.

15. The method of claim 14, wherein the subject is a rodent, and PAG is phenylacetylglycine.

16. The method of claim 15, wherein the PAG in the body fluid sample is measured and determined by NMR spectrometry.

17. The method of claim 14, wherein the subject is a human, and PAG is phenylacetylglutamine.

18. The method of claim 14, wherein the metabolic disorder is selected from the group consisting of drug-induced phospholipidosis, metabolic disorders caused by inborn errors or inherited metabolic disorders.

19. A method for determining risk of mitochondrial toxicity associated with drug-induced phospholipidosis, comprising
a) obtaining a body fluid sample from an animal treated with a phospholipidotic compound,
b) measuring and determining the level of PAG in the body fluid sample, wherein the PAG is phenylacetylglycine, phenylacetylglutamine or equivalents thereof,
c) comparing the level of the determined PAG in the body fluid sample with the level of PAG in a control, and
d) determining whether the animal has a risk of drug-induced phospholipidosis based on an increased level of PAG in the body fluid sample in comparison with the level of PAG in the control, wherein the increased level of PAG in the body fluid sample is indicative that said suspected or known phospholipidotic compound induces mitochondrial toxicity which is associated with drug-induced phospholipidosis.

20. The method of claim 19, wherein the subject is a rodent, and PAG is phenylacetylglycine.

21. The method of claim 20, wherein the PAG in the body fluid sample is measured and determined by NMR spectrometry.

22. The method of claim 19, wherein the subject is a human, and PAG is phenylacetylglutamine.

23. A kit for determining the risk of a phospholipidotic compound for inducing mitochondrial toxicity which is associated with a metabolic disorder, comprising a means or agent for measuring the amount of PAG, wherein the PAG is either phenylacetylglutamine or phenylacetylglycine, and further comprising a user's manual for interpreting the results of any measurement with respect to determining the risk of a phospholipidotic compound inducing mitochondrial toxicity associated with a metabolic disorder.
